(19) United States
(12) Patent Application Publication
(10) Pub. No.: US 2022/0137037 A1
(43) Pub. Date: May 5, 2022

(54) DEVICE AND METHOD FOR DETECTING PROTEIN-BASED MARKER, AND METHOD FOR MANUFACTURING CHIP

(71) Applicant: BOE TECHNOLOGY GROUP CO., LTD., Beijing (CN)

(72) Inventors: Jiapeng WANG, Beijing (CN); Ding DING, Beijing (CN)

(21) Appl. No.: 17/332,812

(22) Filed: May 27, 2021

(30) Foreign Application Priority Data
Oct. 30, 2020 (CN) 202011197985.3

(51) Int. Cl. G01N 33/543; G01N 33/53; G01N 33/58

(52) U.S. Cl. CPC G01N 33/54333; G01N 33/581; G01N 33/5302

(57) ABSTRACT

A detection device and method for detecting a protein-based marker, and method for manufacturing a chip are provided. The detection device includes a first cover plate, in which a liquid inlet and a liquid outlet are provided; a second cover plate attached to the first cover plate to form a chamber; and a chip in the chamber. The chip includes a glass substrate and a micro-hole array layer on a side of the glass substrate. The micro-hole array layer includes a plurality of micro-holes arranged in an array, with each of the micro-holes being a nano micro-hole. The liquid inlet and the liquid outlet are configured such that a solution containing a plurality of magnetic particles enters the chamber via the liquid inlet, flows through at least a portion of the plurality of micro-holes of the micro-hole array layer, and discharges from the liquid outlet.

### CROSS-REFERENCE TO RELATED APPLICATIONS

The present disclosure claims the priority to Chinese Patent Application No. 202011197985.3, filed on Oct. 30, 2020, the contents of which are incorporated herein by reference in its entirety.

### TECHNICAL FIELD

The present disclosure relates to the field of chip technology, in particular to a detection device and method for detecting a protein-based marker, and method for manufacturing a chip.

### BACKGROUND

In the current field of IVD (In Vitro Diagnostic Products, which refers to medical instruments, in vitro diagnostic reagents, and drugs), detection for protein-based markers mainly include enzyme linked immunosorbent assay (ELISA), fluorimetry, and chemiluminescent immunoassay. The chemiluminescent immunoassay is a detection technology with leading sensitivity, precision and accuracy among existing immunodiagnosis methods. However, since the chemiluminescent immunoassay cannot realize the detection of single protein molecule, the detection sensitivity of the existing chemiluminescent immunoassay is close to the theoretical detection limit level, at the present day when the equipment automation technology has developed to a bottleneck.

### SUMMARY

As an aspect, a device for detecting a protein-based marker is provided. The device includes a first cover plate, in which a liquid inlet and a liquid outlet are provided; a second cover plate attached to the first cover plate to form a chamber; and a chip in the chamber. The chip includes a glass substrate and a micro-hole array layer on a side of the glass substrate, the micro-hole array layer including a plurality of micro-holes arranged in an array, with each of the micro-holes being a nano micro-hole. The liquid inlet and the liquid outlet are configured such that a solution containing a plurality of magnetic particles enters the chamber via the liquid inlet, flows through at least a portion of the plurality of micro-holes of the micro-hole array layer, and discharges from the liquid outlet.

In an embodiment, each of the plurality of micro-holes is capable of containing one magnetic particle. Each of the plurality of micro-holes has a diameter in a range from 4 μm to 5 μm, and each of the plurality of micro-holes has a depth in a range from 3 μm to 5 μm.

In an embodiment, the micro-hole array layer includes a photoresist layer on the side of the glass substrate, the photoresist layer having a plurality of initial micro-holes provided therein, and a passivation layer on a side of the photoresist layer away from the glass substrate and covering bottom walls and side walls of the plurality of initial micro-holes of the photoresist layer to form the plurality of micro-holes.

In an embodiment, the liquid inlet and the liquid outlet are respectively at both ends of a diagonal line of the chip.

In an embodiment, the device further includes a magnetic field generator. The magnetic field generator is on a side of the second cover plate away from the first cover plate and configured to form a magnetic field at the micro-hole array layer such that each of the plurality of micro-holes is capable of having a respective one magnetic particle fallen therein under the influence of the magnetic field.

In an embodiment, the first cover plate has a non-patterned planar shape, a groove is provided in the second cover plate, and the first cover plate covers the groove of the second cover plate to form the chamber together with the second cover plate.

In an embodiment, the second cover plate further has a connection region surrounding the chamber and being attached to the first cover plate, and the first cover plate is adhered to the second cover plate with an adhesive in the connection region.

In an embodiment, each of the first cover plate and the second cover plate is made of a material of organic glass, and the adhesive is an ultraviolet adhesive.

In an embodiment, each of the first cover plate and the second cover plate has a non-patterned planar shape, the device further includes a connection layer between the first cover plate and the second cover plate and defining the chamber, and an orthographic projection of the connection layer on the second cover plate does not overlap an orthographic projection of the chip on the second cover plate.

In an embodiment, upper and lower surfaces of the connection layer are bonded to the first cover plate and the second cover plate by plasma bonding.

In an embodiment, each of the first cover plate and the second cover plate is made of a material of inorganic glass, and the connection layer is made of a material of polydimethylsiloxane.

In an embodiment, the passivation layer has a thickness in a range of 2500 Å to 3500 Å.

In an embodiment, a size of the micro-hole array layer along a direction parallel to the glass substrate is one third to one half of a size of the glass substrate along the direction.

As another aspect, a detection device configured to detect a protein-based marker, comprising: a first cover plate, in which a liquid inlet and a liquid outlet are provided; a second cover plate attached to the first cover plate to form a chamber; and a chip in the chamber and comprises a glass substrate and a micro-hole array layer on a side of the glass substrate, the micro-hole array layer comprising a plurality of micro-holes arranged in an array, with each of the micro-holes being a nano micro-hole; and a magnetic field generator on a side of the second cover plate away from the first cover plate. The liquid inlet and the liquid outlet are respectively located at both ends of a diagonal line of the chip, and are configured such that a solution containing a plurality of magnetic particles enters the chamber via the liquid inlet, flows through at least a portion of the plurality of micro-holes of the micro-hole array layer, and discharges from the liquid outlet. The magnetic field generator is configured to form a magnetic field at the micro-hole array layer such that each of the plurality of micro-holes is capable of having a respective one magnetic particle fallen therein under the influence of the magnetic field. The micro-hole array layer includes a photoresist layer on the side of the glass substrate, the photoresist layer having a plurality of initial micro-holes provided therein, and a passivation layer on a side of the photoresist layer away from the glass substrate and covering bottom walls and side walls of the plurality of initial micro-holes of the photoresist layer to form the plurality of micro-holes.

Yet another aspect, a method for manufacturing a chip for detecting a protein-based marker is provided. The method includes providing a glass substrate; and forming a micro-hole array layer on the glass substrate. The micro-hole array layer includes a plurality of micro-holes arranged in an array, with each of the micro-holes being a nano micro-hole.

In an embodiment, forming the micro-hole array layer on the glass substrate, comprises: forming a photoresist layer on a side of the glass substrate; patterning the photoresist layer to form a plurality of initial micro-holes arranged in an array on the photoresist layer; and forming a passivation layer on a side of the patterned photoresist layer away from the glass substrate such that the passivation layer covers bottom walls and sidewalls of the plurality of initial micro-holes of the patterned photoresist layer to form the plurality of micro-holes. Each of the plurality of micro-holes has a diameter in a range from 4 μm to 5 μm, and each of the plurality of micro-holes has a depth in a range from 3 μm to 5 μm.

Yet another aspect, a method for detecting a protein-based marker is provided. The method includes reacting magnetic particles coupled with an antibody of the protein-based marker with a solution to be tested and a solution containing a labeled antibody in sequence to obtain labeled magnetic particles; loading all of the magnetic particles from the liquid inlet onto the detection device, such that at least a portion of the magnetic particles fall into at least a portion of the micro-holes of the plurality of micro-holes, and each of the plurality of micro-holes is capable of having a respective one magnetic particle fallen therein; and determining an amount of the protein-based marker in the solution to be tested by detecting a total amount of magnetic particles falling into the at least a portion of the micro-holes and an amount of the labeled magnetic particles.

In an embodiment, after the at least a portion of the magnetic particles fall into the at least a portion of the micro-holes of the plurality of micro-holes, the method further includes removing magnetic particles not falling into a micro-hole by a method of an oil phase flushing.

In an embodiment, the removing the magnetic particles not falling into the micro-hole by the method of the oil phase flushing, includes flushing the chip by using an electronic fluoride solution with a volume in a range from 40 μL to 60 μL and a flow rate in a range from 2 μL/s to 4 μL/s.

In an embodiment, the removing the magnetic particles not falling into the micro-hole by the method of the oil phase flushing, includes flushing the chip by using an electronic fluoride solution with a volume of 50 μL and a flow rate of 3 μL/s.

### BRIEF DESCRIPTION OF THE DRAWINGS

FIG. 1-1 is a top view showing a chip of a detection device according to an embodiment of the present disclosure;

FIG. 1-2 is a cross-sectional view showing a chip of a detection device according to an embodiment of the present disclosure;

FIG. 1-3 is a cross-sectional view showing a chip of a detection device according to an embodiment of the present disclosure;

FIG. 2 is a flowchart showing a method for manufacturing a chip according to an embodiment of the present disclosure;

FIG. 3 is fluorescent microscope images showing magnetic particles that are dyed and washed for various times according to an embodiment of the present disclosure;

FIG. 4 is fluorescence microscope images showing magnetic particles after being washed by electron fluoride solutions under various volumes and flow rates according to an embodiment of the present disclosure; and

FIG. 5 is fluorescence microscope images showing detection results for solutions to be tested with various concentrations according to an embodiment of the present disclosure.

### DETAILED DESCRIPTION

In order to make those skilled in the art better understand the technical solutions of the present disclosure, the present disclosure is further described in detail below with reference to the accompanying drawings and specific embodiments.

In an embodiment, after studying and analyzing the technology for detecting a protein-based marker by using magnetic particles as a solid phase carrier, it is found that in the detection technology using magnetic particles as the solid phase carrier, the magnetic particles have a larger surface area than a two-dimensional surface, which helps realization of sufficiently reaction during the detection process. However, a PH value of a buffer solution in which the magnetic particles are located and concentrations of various ions in the buffer solution may affect the charged magnetic particles (e.g., a phenomenon of agglomeration occurs). If the magnetic particles are distributed uniformly, such a problem can be solved, a more accurate signal can be obtained, and then more accurate detection on the protein-based marker can be realized. For example, the antibody-labeled magnetic particles may be attached to a polydimethylsiloxane (PMDS)-based male mold by heating PDMS. However, this method requires heating the mold, which can easily cause protein denaturation, which in turn leads to inactivation of antigen and antibody, resulting in a significant decrease in detection precision; and PDMS as a microarray material is prone to aging and denaturation, has a short service life and a high cost, and the like.

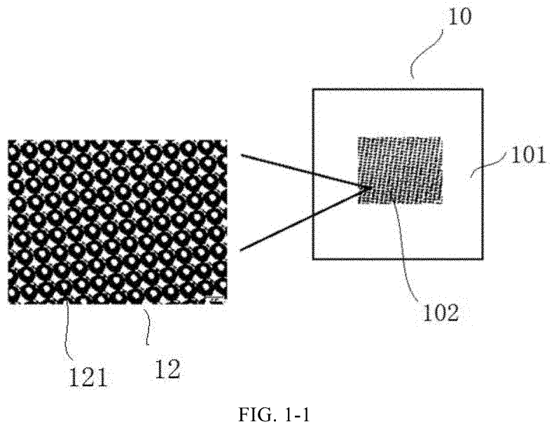

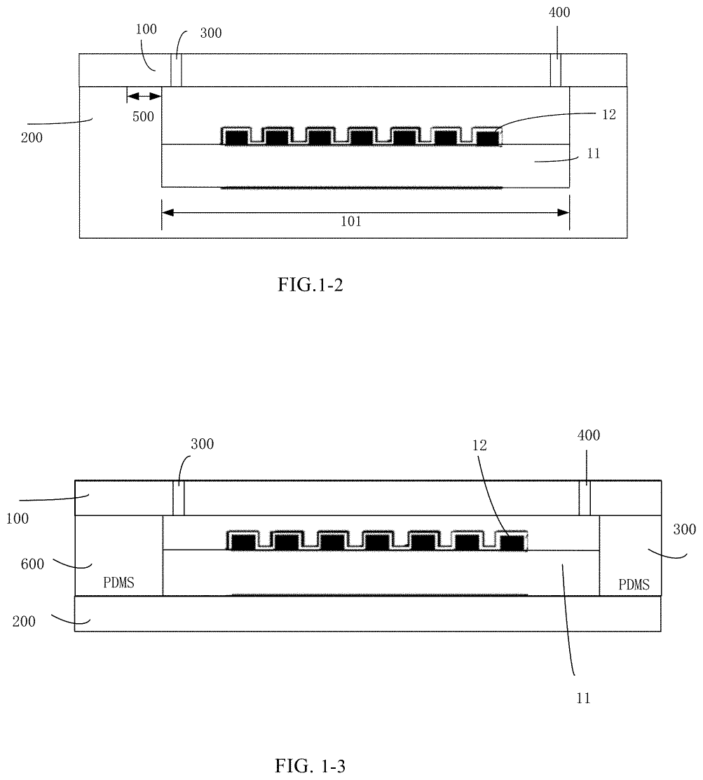

In view of the above problems, the present embodiment provides a detection device for detecting a protein-based marker. As shown in FIG. 1-1, FIG. 1-2, FIG. 1-3 and FIG. 2, the detection device includes a chip 10, a first cover plate 100, and a second cover plate 200. The chip 10 includes a glass substrate 11 and a micro-hole array layer 12 on the glass substrate 11, and the micro-hole array layer 12 is stacked on the glass substrate 11. The micro-hole array layer 12 may include a plurality of micro-holes 121. The first cover plate 100 and the second cover plate 200 are bonded to form a chamber, and the chip 10 is located in the chamber. A liquid inlet 300 and a liquid outlet 400 are provided in the first cover plate 100. Both of the liquid inlet 300 and the liquid outlet 400 are located in a region where the first cover plate is bonded to the chip 10 or a region where the first cover plate overlaps the chip 10, and both of the liquid inlet 300 and the liquid outlet 400 are communicated with the plurality of micro-holes 121 of the micro-hole array layer 12. The liquid inlet 300 and the liquid outlet 400 may be configured such that a solution entering through liquid inlet 300 may enter the chamber, flow through the micro-holes 121 in the micro-hole array layer 12, and finally exit from the liquid outlet 400.

The detection device according to the present embodiment includes the chip 10 having the micro-hole array layer 12 and the glass substrate 11, the first cover plate 100, and the second cover plate 200. The chip 10 may be assembled in the chamber enclosed by the first cover plate 100 and the second cover plate 200 which are bonded together. In this configuration, a buffer solution having magnetic particles to be reacted with a solution to be tested is injected from the liquid inlet 300 of the first cover plate 100 and flows on the chip 10, such that the magnetic particles uniformly fall into each of the micro-holes 121 of the micro-hole array layer 12. By matching a size of the magnetic particle with a size of the micro-hole 121, each of the micro-holes 121 may contain only one magnetic particle. As a result, an amount of the protein-based marker in the solution to be tested may be accurately calculated based on the Poisson ratio according to a total amount of the magnetic particles that fall into the micro-holes 121 and an amount of the magnetic particles undergoing the immunoreaction. Since the micro-holes 121 are directly formed in the micro-hole array layer 12 of the chip 10 employing the glass substrate 11, heating is not required any more, thereby not denaturing the protein, ensuring the detection precision of the protein-based marker, and realizing protein detection with a concentration level of pg/mL (10⁻¹²). Furthermore, the glass-based chip 10 has a strong corrosion resistance, is not prone to aging or denaturation, and has a long service life and a low cost.

The micro-hole array layer 12 of the chip 10 may include a photoresist layer 14 and a passivation layer 13. The photoresist layer 14 may be located on the glass substrate 11 and stacked on the glass substrate 11. A plurality of initial micro-holes 141 may be formed in the photoresist layer 14. The passivation layer 13 may cover the photoresist layer 14 and cover bottom walls and sidewalls of the plurality of initial micro-holes 141 to form the plurality of micro-holes 121. The passivation layer 13 is a hydrophilic modification layer made of silicon nitride, and the molecular acting force between the passivation layer and the protein is small, thereby decreasing the adsorption of the protein, improving the fluidities of the buffer solution and the magnetic particles in the buffer solution, preventing a structure of the magnetic particles undergoing the immunoreaction from being damaged, and improving a filling rate of the magnetic particles in the micro-holes and the detection precision of detecting the protein-based marker.

In an embodiment, the process for forming the micro-hole array layer 12 may include: 1) spin coating a photoresist layer 14 on the glass substrate 11 (e.g., spin coating with a speed of 400 rpm for 30 seconds, and baked at 110° C. for 150 seconds, and the photoresist layer 14 may have a thickness of 4 micrometers); 2) exposing and developing the photoresist layer 14 by using a mask (e.g., development for 70 seconds, and exposure for 4500 milliseconds) to obtain the plurality of initial micro-holes 141 arranged in an array; 3) depositing the passivation layer 13 by using a Plasma Enhanced Chemical Vapor Deposition (PECVD) process. The passivation layer 13 may have a thickness greater than or equal to 2500 Å and less than or equal to 3500 Å, so as to space the buffer solution, by the passivation layer 13, apart from the glass substrate 11 and the photoresist layer 14, thereby reducing the adsorption of the protein. In an embodiment, the passivation layer 13 may have a thickness of 3000 Å.

It should be noted that the above structure of the chip 10 is described in one embodiment of the present embodiment, and the structure of the chip 10 is not limited to the embodiment, as long as the chip 10 has the glass substrate 11 and the micro-hole array layer 12 and a plurality of micro-holes 121 arranged in an array are formed in the micro-hole array layer 12.

Each of the first cover plate 100 and the second cover plate 200 is a transparent organic glass substrate to facilitate observation of the magnetic particles. In an embodiment, when the first cover plate 100, the second cover plate 200 and the chip 10 are assembled, the first cover plate 100 and the second cover plate 200 may be aligned with each other, and alternatively, the first cover plate 100 and the second cover plate 200 may be not aligned with each other or may be staggered with each other, as long as the chip 10 is packaged between the two cover plates 100 and 200, which is not limited to the embodiments of the present disclosure. The specific materials of the two cover plates may be the same or different, which is not limited to the embodiment. In an embodiment, the first cover plate and the second cover plate may be made of two different materials.

For the second cover plate 200 on a side of the chip 10 away from the micro-holes 121, the second cover plate may employ a material that is easily attached or fixed to the chip 10 without considering the fluidity of the buffer solution; and for the first cover plate 100 located on a side of the chip 10 proximal to the micro-holes 121, the fluidity of the buffer solution needs to be considered, that is, it is not only necessary to ensure that the chip 10 and the first cover plate 100 can be bonded and fixed, but also to ensure the fluidity of the buffer solution, so that the selected material of the first cover plate 100 should not obstruct the flow of the buffer solution.

In an embodiment, as shown in FIG. 1-2, the chamber is formed in the second cover plate 200. The second cover plate 200 may further include a connection region 500 surrounding the chamber. The second cover plate 200 may be attached to the first cover plate 100 in the connection region 500, so that the chip 10 may be limited in the chamber with closed side surfaces, so as to prevent the position of the chip 10 from moving during the detection process, which may result in decreased accuracy of the detection result, and the like.

In an embodiment, the first cover plate 100 has a planar shape that is not patterned. A groove is formed in the second cover plate 200, and the first cover plate 100 covers the groove of the second cover plate 200 to form the chamber together with the second cover plate 200.

As shown in FIG. 1-1, the chip 10 may include an array region 102 and a bonding region 101 around the array region 102. The micro-hole array layer 12 is formed in the array region 102. The bonding region 101 is disposed around the array region 102, so that the chip 10 is connected to the first cover plate 100 and the second cover plate 200, which can ensure the bonding effect of the chip 10, and ensure that the buffer solution uniformly flows through the array region 102 and each micro-hole 121 falls into only one magnetic particle therein. A size of the array region 102 (i.e., the micro-hole array layer 12) along a direction parallel to the glass substrate may be one third to one half of a size of the glass substrate 11 along the direction, so as to ensure an enough area of the bonding region 101 and close bonding between the chip 10 and the two cover plates. For example, the chip 10 may be a glass plate with an area of 10 mm×10 mm, an array region 102 with an area of 4 mm×4 mm is inside the chip 10, and 500×500 micro-holes 121 are in the array region 102.

Further, as shown in FIG. 1-2, an adhesive, such as an ultraviolet (UV) adhesive, may be disposed between the second cover plate 200 and the first cover plate 100 in the connection region 500, and the chip 10 may be adhered and bonded to the two cover plates with the adhesive.

In another embodiment, the detection device further includes a connection layer 600 between the first cover plate 100 and the second cover plate 200 for defining the chamber. Each of the first cover plate and the second cover plate has a non-patterned planar shape and is made of inorganic glass material. The connection layer 600 is made of polydimethylsiloxane (PDMS) material. Upper and lower surfaces of the connection layer 600 are bonded to each of the first cover plate 100 and the second cover plate 200 by plasma bonding (e.g., plasma is generated by ionizing air through high-voltage discharge, and is blown out by air flow. Under the action of the plasma, physical and chemical changes take place on a surface of the material to be treated, which makes the surface clean and smooth for further processing). An orthographic projection of the connection layer 600 on the second cover plate 200 does not overlap an orthographic projection of the chip 10 on the second cover plate 200. With the plasma bonding, in a contact region 500 where the connection layer 600 is in contact with each of the first cover plate 100 and the second cover plate 200 (i.e., as shown in FIG. 1-3, the entire contact surface on which the connection layer 600 is contact with each of the first cover plate 100 and the second cover plate 200), acting forces, at various positions of the connection layer 600, between the connection layer 600 and the first and second cover plates 100, 200 are substantially equal to each other, so that the first and second cover plates 100, 200 and the connection layer 600 are bonded more uniformly and stably.

It should be noted that the above-mentioned encapsulations of the chip 10 between the two cover plates 100 and 200 are only two optional implementations of the present embodiment, and the present disclosure is not limited thereto. For example, two cover plates each having a flat-plate structure may be employed, the two cover plates may be respectively attached to upper and lower surfaces of the chip 10 and be bonded together in the bonding region 101 of the chip, so as not to affect the flow of the buffer solution containing the magnetic particles.

In order to realize that the buffer solution fully flows through each of the micro-holes 121 of the micro-hole array layer 12 and only one magnetic particle falls into one corresponding micro-hole 121, the liquid inlet and the liquid outlet may be disposed in the first cover plate in the bonding region 101, and respectively located at two opposite vertex angles of the chip 10 or respectively located at two ends of a diagonal line of the chip 10, so that the buffer solution enters the chamber from the liquid inlet, flows through each of the micro-holes 121 from one corner of the chip 10, and the remaining buffer solution is discharged from the liquid outlet at the opposite corner, thereby ensuring that almost each of the micro-holes 121 only has one respective magnetic particle fallen therein.

In order to make each micro-hole 121 be capable of having only one magnetic particle falling into it, a size of the magnetic particle can be designed to match a size of the micro-hole 121. For example, a depth or a diameter of the micro-hole 121 is larger than a diameter of the magnetic particle and smaller than twice the diameter of the magnetic particle. For example, considering processing accuracy and observability, a diameter of the micro-hole 121 may be equal to or greater than 4 μm, and smaller than or equal to 5 μm, preferably 4.5 μm; a depth of the micro-hole 121 may be equal to or greater than 3 μm, and smaller than or equal to 5 μm, preferably 4 μm. In this case, a diameter of the magnetic particle may be greater than 2.5 μm and less than 3 μm.

In order to facilitate the magnetic particles to enter the micro-holes 121, the detection device may further include a magnetic field generator at a periphery region of the chip 10, for example, located at a side of the second cover plate 200 away from the first cover plate 100. The magnetic field generator is configured to generate a magnetic field in the micro-hole array layer 12 (i.e., the array region 102), with a direction of the magnetic field being perpendicular to the glass substrate 11, so that the magnetic substances (i.e., magnetic particles) falling into the micro-holes 121 are subjected to a force toward the bottom of the micro-holes 121.

Based on the same concept of the detection device, the present embodiment further provides a method for manufacturing the chip 10, as a component of the detection device, for detecting a protein-based marker, and the method includes: (1) providing a glass substrate 11, such as a transparent glass substrate 11, which facilitates observation of the magnetic particles; (2) forming a micro-hole array layer 12 on the glass substrate 11. The micro-hole array layer 12 includes a plurality of micro-holes 121 arranged in an array for separating the magnetic particles during a detection process of the protein-based marker.

As above, the step of forming the micro-hole array layer 12 on the glass substrate 11 may include: (21) coating a photoresist layer 14 on the grass substrate, wherein the photoresist layer 14 may be used for forming the plurality of micro-holes 121 arranged in an array; (22) performing exposure and development on the photoresist layer 14 to form a plurality of initial micro-holes 141 arranged in an array on the photoresist layer 14; and (23) forming a passivation layer 13 on the exposed and developed photoresist layer 14, wherein the passivation layer 13 covers bottom walls and side walls of the plurality of initial micro-holes 141 to form the plurality of micro-holes 121 arranged in an array.

The method for manufacturing the chip 10 according to the present embodiment can manufacture the chip 10 including the glass substrate 11 and the micro-hole array layer 12. The chip 10 can be applied to the above-mentioned detection device for separating the magnetic particles, which improves detection accuracy, and the like.

Based on the same concept of the above detection device, an embodiment further provides a method for detecting a protein-based marker, which can be applied to a solution to be tested containing the protein-based marker, and the method may include steps S1 to S3.

At step S1, the magnetic particles coupled with an antibody of a protein-based marker are reacted with the solution to be tested and a solution containing a labeled antibody in sequence to obtain the magnetic particles with a label.

During the detection process, the magnetic particles coupled with the antibody of protein-based marker are added into the solution to be tested, so that the antibody coupled with the magnetic particles is combined with a protein-based antigen in the solution to be tested. And then all the magnetic particles (including the magnetic particles combined with the antigen) are washed and added into the solution containing the labeled antibody for reaction, so that the antigen combined with the magnetic particles is reacted with the labeled antibody to obtain the magnetic particles with the label.

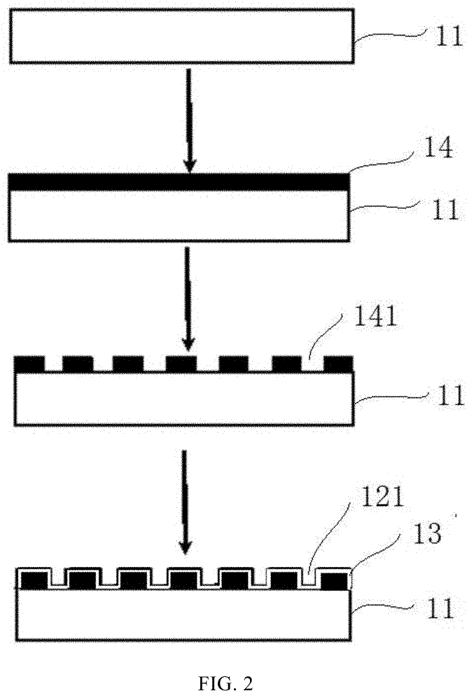

In an embodiment, before the magnetic particles undergo the immunoreaction, the magnetic particles may be dyed to obtain fluorescent magnetic particles, which facilitates observation of the magnetic particles. After being dyed, the dyed magnetic particles may be washed to minimize the background fluorescence, so that the influence of the dyeing solution on subsequent detection can be avoided. Before the magnetic particles are used, the magnetic particles may be placed in a preservation solution for preservation, so as to ensure the biological activity of the magnetic particles. Furthermore, in the embodiment, an experiment is performed with regarding to the number of times of washing the dyed magnetic particles to obtain an optimal number of times of washing by which not only the background fluorescence can be minimized, but also the fluorescent effect of the magnetic particles can be ensured. As shown in FIGS. 3, (A), (B), and (C) are images observed by a fluorescent microscope after the magnetic particles are washed for three times, four times, and five times, respectively. Bright dots in the images may represent the fluorescent magnetic particles. As can be seen from FIG. 3, after the magnetic particles are washed for five times, the background fluorescence of the magnetic particles that do not fall into the micro-holes can be minimized while the fluorescent effect of the magnetic particles can be ensured.

At step S2, the chip 10 is manufactured by using above method for manufacturing the chip 10, and the detection device is manufactured based on the resultant chip 10, the first cover plate and the second cover plate, which can refer to above embodiment of manufacturing method and will not be described herein again.

At step S3, all of the magnetic particles are loaded from the liquid inlet onto the detection device, so that at least a portion of the magnetic particles fall into at least a portion of the plurality of micro-holes 121. An amount of the protein-based marker in the solution to be tested may be determined by detecting the total amount of the magnetic particles falling into the micro-holes 121 and the amount of the labeled magnetic particles.

The loading process may refer to the operating process of the above-mentioned detection device, that is, the buffer solution containing the magnetic particles is injected from the liquid inlet of the first cover plate, and the remaining buffer solution and remaining magnetic particles are discharged from the liquid outlet, such that the magnetic particles can fall into each of the micro-holes 121 during the flowing of the buffer solution.

It should be noted that the execution sequence of step S1 and step S2 is not particularly limited in the present embodiment, and the two steps may be performed simultaneously.

In order to observe the magnetic particles and detect the number of the magnetic particles, after the at least a portion of the magnetic particles fall into the at least a portion of the plurality of micro-holes 121, the method may further includes: removing the magnetic particles that do not fall into the micro-hole 121 by oil phase flushing, so as to avoid the magnetic particles that do not fall into the micro-hole 121 from affecting the observation and detection of the magnetic particles that already fall into the micro-holes 121 (e.g., it is impossible to distinguish whether the magnetic particles fall into the micro-holes 121 or not).

In an embodiment, the step of removing the magnetic particles that do not fall into the micro-hole 121 by oil phase flushing may include: flushing the detection device by using an electronic fluoride solution with a volume in a range from 40 μL to 60 μL and a flow rate in a range from 2 μL/s to 4 μL/s, so as to remove the magnetic particles that do not fall into the micro-holes 121.

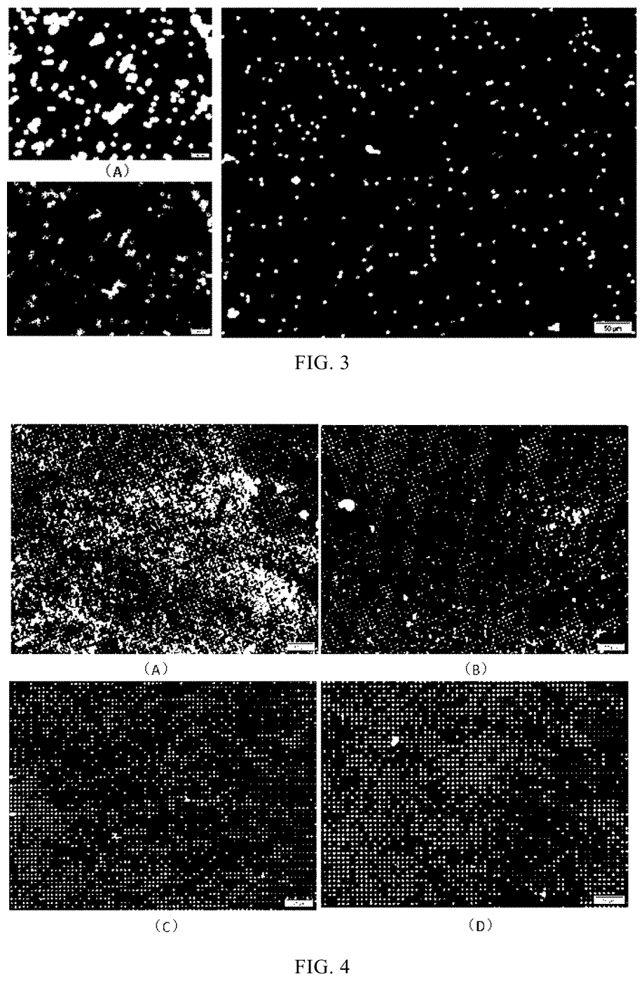

In the embodiment, a comparative experiment is performed on the washing effect at various volumes and flow rates, before the volume and flow rate of the oil phase liquid are determined. As shown in FIG. 4, when the solution to be tested with a volume of 15 μL it and containing 500000 magnetic particles flows over the chip 10 having 200000 micro-holes 121, fluorescence microscope images (in which the bright dots in the (A)-(D) of FIG. 4 represent the magnetic particles) are observed by a fluorescence microscope with a 10-fold objective lens at an excitation wavelength of 495 nm and an exposure time of 700 ms. (A) of FIG. 4 is an image showing the magnetic particles do not undergo oil phase flushing; (B) of FIG. 4 is an image showing the magnetic particles washed by an electronic fluoride solution with a volume of 30 μL at a flow rate of 10 μL/s; (C) of FIG. 4 is an image showing the magnetic particles washed by an electronic fluoride solution with a volume of 50 μL at a flow rate of 5 μL/s; and (D) of FIG. 4 is an image showing the magnetic particles washed by an electronic fluoride solution with a volume of 50 μL at a flow rate of 3 μL/s. As shown in FIG. 4, flushing the chip 10 by using an electronic fluoride solution with a volume of 50 μL and a flow rate of 3 μL/s can minimize the noise while keeping more magnetic particles in the micro-holes.

The immunoassay method will be described in detail below with reference to specific examples.

1. Manufacture of the Detection Device

Referring to the manufacturing method of the chip 10 and the structure of the detection device, the chip 10 with an area of 10 mm×10 mm is manufactured, and the detection device is manufactured based on the resultant chip 10, which is not described herein again.

2. Preparation of Reagent

According to the digital immunoassay, a solution containing magnetic particles is required to be applied to the chip 10, so that a single magnetic particle falls into a respective one of the micro-holes 121 of the array for separation and observation. The condition under which the magnetic particles fall into the micro-holes is related to a concentration of the solution containing the magnetic particles and the waiting time after the solution containing magnetic particles is applied to the chip 10. For example, in a case where the chip 10 is formed with 250000 micro-holes thereon, if the enzyme-catalyzed fluorescence response observed from 100 magnetic particles is within the acceptable Poisson noise (i.e., less than or equal to 10%), and a ratio of the magnetic particles with enzyme fluorescence response to all the magnetic particles falling into the micro-holes 121 is 1% as the lower limit of the detection, it can be calculated that the minimum value of proportion of the magnetic particles entering micro-holes is 4.6% according to the Poisson formula, that is, the minimum value of the concentration of the magnetic particles undergoing the immunoreaction may be in a range from 4000 μL to 7000/μL.

Specifically, carboxyl magnetic particles with a diameter of 2.8 μm are reacted with and activated by carbodiimide (EDC, as an activator), and then are reacted and coupled with a SPRN-5 capture antibody (i.e., an antibody of bovine serum albumin (BSA)). After the antibody is coupled to the magnetic particles, the magnetic particles are required to be dyed for subsequent calculation, so that the number of all the magnetic particles (including all the magnetic particles coupled and not coupled to the antigen to be tested) falling into the micro-holes can be directly determined.

The concentration of the dye for dyeing the magnetic particles is 10 mg/mL (1 mg of dye is dissolved in a diluent with a volume of 100 μL). 10 million magnetic particles coupled with capture antibody are suspended in a NaHCO3 solution (0.1 Mol) with a volume of 600 μL, and the dye with a volume of 4.4 μL and pH value of 8.5 is added into the NaHCO3 solution. The dyeing may be carried out at room temperature with oscillating for 2 hours. The dyed magnetic particles may be washed for five times with PBST (i.e., 0.1% of Tween-20). At last, the dyed magnetic particles coupled with the antibody may be stored in a magnetic particle storage solution, and the magnetic particle storage solution may contain 50 mMol of buffer solution with the pH value of 7.8, 150 mMol of NaCl, 10 mMol of EDTA, 1% of BSA, 1% of Triton-100, 0.15% of Proclin-100 and the like.

3. Specific Immune Reaction

The sample to be tested is selected from diluents, of standard solutions, with a volume of 100 μL, and containing PSA antigen with various concentrations, and the diluents is PBST (i.e., 0.1% of Tween-20) added with 2.5% of BSA solution. 500000 magnetic particles are required for a single reaction and added into the diluent of the sample to be tested for immunoreaction reaction at room temperature for 2 hours with continuous oscillation. After the reaction is finished, the magnetic particles are washed with a washing solution (i.e., PBST) for five times. The washed magnetic particles are added into a solution with a volume of 50 μL and a concentration of 1 nMol containing a labeled antibody for immunoreaction reaction at room temperature for one hour with continuous oscillation. After the reaction is finished, the magnetic particles are washed with the washing solution for three times. A streptavidin-β-galactosidase solution (in which 40 pMol of PBST is a solvent and 1 mMol of MgCl₂ is an additive) is added, so that the magnetic particles react with the enzyme-labeled antibody at room temperature for 30 minutes. After the reaction is finished, the magnetic particles are washed for five times. Finally, the resultant magnetic particles are added to 100 uMol of a substrate with a volume of 15 μL and then introduced onto the chip 10 of the detection device.

4. Flushing Process After the Fluorescent Magnetic Particles are Introduced

After the solution containing the magnetic particles is loaded onto the chip 10, a magnetic field may be applied from outside of the chip 10 to allow the magnetic particles to fall into the micro-holes 121. Meanwhile, a large amount of disordered magnetic particles that do not enter the micro-holes 121 may be deposited on the chip 10, which affects the collection of the signal. To solve this problem, the detection device (i.e., at the position where the chip 10 is located) can be flushed with an electron fluoride solution with a volume of 50 μL at a flow rate of 3 μL/s in order to minimize the noise while maintain more magnetic particles in the micro-holes 121, thereby ensuring the detection accuracy.

5. The Result of Detection

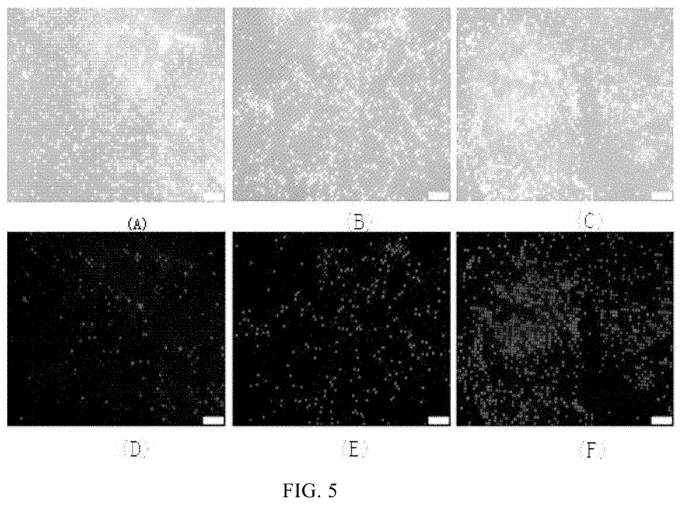

Various concentrations of antigens to be tested such as 0 pg/mL (i.e., a negative sample), 0.2 pg/mL, and 1000 pg/mL are respectively used for detection. The detection result may be observed through a fluorescence microscope equipped with a 10-megapixel camera. First, an image of fluorescence emission light with a wavelength of 520 nm is obtained at an excitation light with a wavelength of 495 nm for determining the positions of all of the magnetic particles in the micro-holes 121, as shown in (A), (B), and (C) of FIG. 5. And then, an image of fluorescence emission light with a wavelength of 620 nm is obtained at an excitation light with a wavelength of 577 nm for determining the fluorescence response of the magnetic particles coupled with the antigen and labeled antibody, as shown in (D), (E), and (F) of FIG. 5. The ratio of the magnetic particles coupled with the antigen to all the magnetic particles may be calculated based on the Poisson formula, thereby realizing high-precision digital detection on the protein-based marker and qualitative observation and detection on various concentrations of the substance to be tested.

In conclusion, the immunoassay according to the embodiment can ensure the precision and accuracy of detection on the protein-based marker, and the protein detection with the pg/mL (10⁻¹²) concentration level can be realized. The detection method is easy to carry out and has strong implementability, and the like.

It should be understood that the above implementations are merely exemplary embodiments for the purpose of illustrating the principles of the present disclosure, however, the present disclosure is not limited thereto. It will be apparent to those skilled in the art that various changes and modifications can be made without departing from the spirit and essence of the present disclosure, which are also to be regarded as the protection scope of the present disclosure.

What is claimed is:

1. A device for detecting a protein-based marker, comprising:
a first cover plate, in which a liquid inlet and a liquid outlet are provided;
a second cover plate attached to the first cover plate to form a chamber; and
a chip in the chamber, wherein
the chip comprises a glass substrate and a micro-hole array layer on a side of the glass substrate, the micro-hole array layer comprising a plurality of micro-holes arranged in an array, with each of the micro-holes being a nano micro-hole, and
the liquid inlet and the liquid outlet are configured such that a solution containing a plurality of magnetic particles enters the chamber via the liquid inlet, flows through at least a portion of the plurality of micro-holes of the micro-hole array layer, and discharges from the liquid outlet.

2. The device according to claim 1, wherein
each of the plurality of micro-holes is capable of containing one magnetic particle,
each of the plurality of micro-holes has a diameter in a range from 4 μm to 5 μm, and
each of the plurality of micro-holes has a depth in a range from 3 μm to 5 μm.

3. The device according to claim 1, wherein the micro-hole array layer comprises:
a photoresist layer on the side of the glass substrate, the photoresist layer having a plurality of initial micro-holes provided therein, and
a passivation layer on a side of the photoresist layer away from the glass substrate and covering bottom walls and side walls of the plurality of initial micro-holes of the photoresist layer to form the plurality of micro-holes.

4. The device according to claim 1, wherein
the liquid inlet and the liquid outlet are respectively at both ends of a diagonal line of the chip.

5. The device according to claim 1, further comprising a magnetic field generator, wherein
the magnetic field generator is on a side of the second cover plate away from the first cover plate, and
the magnetic field generator is configured to form a magnetic field at the micro-hole array layer such that each of the plurality of micro-holes is capable of having a respective one magnetic particle fallen therein under influence of the magnetic field.

6. The device according to claim 1, wherein
the first cover plate has a non-patterned planar shape,
the second cover plate is provided therein with a groove, and
the first cover plate covers the groove of the second cover plate to form the chamber together with the second cover plate.

7. The device according to claim 6, wherein
the second cover plate further has a connection region surrounding the chamber and being attached to the first cover plate, and
the first cover plate is adhered to the second cover plate with an adhesive in the connection region.

8. The device according to claim 7, wherein
each of the first cover plate and the second cover plate is made of a material of organic glass, and
the adhesive is an ultraviolet adhesive.

9. The device according to claim 1, wherein
each of the first cover plate and the second cover plate has a non-patterned planar shape,
the device further comprises a connection layer between the first cover plate and the second cover plate and defining the chamber, and
an orthographic projection of the connection layer on the second cover plate does not overlap an orthographic projection of the chip on the second cover plate.

10. The device according to claim 9, wherein
upper and lower surfaces of the connection layer are bonded to the first cover plate and the second cover plate by plasma bonding.

11. The device according to claim 10, wherein
each of the first cover plate and the second cover plate is made of a material of inorganic glass, and
the connection layer is made of a material of polydimethylsiloxane.

12. The device according to claim 3, wherein
the passivation layer has a thickness in a range of 2500 Å to 3500 Å.

13. The device according to claim 1, wherein
a size of the micro-hole array layer along a direction parallel to the glass substrate is one third to one half of a size of the glass substrate along the direction.

14. A detection device configured to detect a protein-based marker, comprising:
a first cover plate, in which a liquid inlet and a liquid outlet are provided;
a second cover plate attached to the first cover plate to form a chamber; and
a chip in the chamber and comprises a glass substrate and a micro-hole array layer on a side of the glass substrate, the micro-hole array layer comprising a plurality of micro-holes arranged in an array, with each of the micro-holes being a nano micro-hole; and
a magnetic field generator on a side of the second cover plate away from the first cover plate, wherein
the liquid inlet and the liquid outlet are respectively located at both ends of a diagonal line of the chip, and are configured such that a solution containing a plurality of magnetic particles enters the chamber via the liquid inlet, flows through at least a portion of the plurality of micro-holes of the micro-hole array layer, and discharges from the liquid outlet, and
the magnetic field generator is configured to form a magnetic field at the micro-hole array layer such that each of the plurality of micro-holes is capable of having a respective one magnetic particle fallen therein under influence of the magnetic field, and
the micro-hole array layer comprises:
a photoresist layer on the side of the glass substrate, the photoresist layer having a plurality of initial micro-holes provided therein, and
a passivation layer on a side of the photoresist layer away from the glass substrate and covering bottom walls and side walls of the plurality of initial micro-holes of the photoresist layer to form the plurality of micro-holes.

15. A method for manufacturing a chip for detecting a protein-based marker, comprising:
providing a glass substrate; and
forming a micro-hole array layer on the glass substrate, the micro-hole array layer comprising a plurality of micro-holes arranged in an array, with each of the micro-holes being a nano micro-hole.

16. The method according to claim 15, wherein forming the micro-hole array layer on the glass substrate comprises:
forming a photoresist layer on a side of the glass substrate;
patterning the photoresist layer to form a plurality of initial micro-holes arranged in an array on the photoresist layer; and
forming a passivation layer on a side of the patterned photoresist layer away from the glass substrate such that the passivation layer covers bottom walls and sidewalls of the plurality of initial micro-holes of the patterned photoresist layer to form the plurality of micro-holes, wherein
each of the plurality of micro-holes has a diameter in a range from 4 μm to 5 μm, and
each of the plurality of micro-holes has a depth in a range from 3 μm to 5 μm.

17. A method for detecting a protein-based marker, comprising:
reacting magnetic particles coupled with an antibody of the protein-based marker with a solution to be tested and a solution containing a labeled antibody in sequence to obtain labeled magnetic particles;
loading all of the magnetic particles from the liquid inlet onto the detection device according to claim 1, such that at least a portion of the magnetic particles fall into at least a portion of the micro-holes of the plurality of micro-holes, and each of the plurality of micro-holes is capable of having a respective one magnetic particle fallen therein; and
determining an amount of the protein-based marker in the solution to be tested by detecting a total amount of magnetic particles falling into the at least a portion of the micro-holes and an amount of the labeled magnetic particles.

18. The method according to claim 17, after the at least a portion of the magnetic particles fall into the at least a portion of the micro-holes of the plurality of micro-holes, further comprising:
removing magnetic particles not falling into a micro-hole by a method of an oil phase flushing.

19. The method according to claim 18, wherein the removing the magnetic particles not falling into the micro-hole by the method of the oil phase flushing, comprises:
flushing the chip by using an electronic fluoride solution with a volume in a range from 40 μL to 60 μL and a flow rate in a range from 2 μL/s to 4 μL/s.

20. The method according to claim 19, wherein the removing the magnetic particles not falling into the micro-hole by the method of the oil phase flushing, comprises:
flushing the chip by using an electronic fluoride solution with a volume of 50 μL and a flow rate of 3 μL/s.
